(19) United States
(12) Patent Application Publication
(10) Pub. No.: US 2025/0281310 A1
(43) Pub. Date: Sep. 11, 2025

(54) SYSTEM AND METHODS FOR TREATING A 3D PRINTED PROSTHETIC LINER

(71) Applicant: Quorum Orthopedics, Inc., Windsor, CO (US)

(72) Inventors: Joe Johnson, Windsor, CO (US); John Fleischmann, Windsor, CO (US); Sean Robert McClure, II, Johnstown, CO (US)

(21) Appl. No.: 18/862,489

(22) PCT Filed: May 4, 2023
(86) PCT No.: PCT/US2023/021039

§ 371 Date: Nov. 1, 2024

Related U.S. Application Data
(60) Provisional application No. 63/338,483, filed on May 5, 2022.

(51) Int. Cl. A61F 2/78; B33Y 40/20; B33Y 80/00

(52) U.S. Cl. CPC A61F 2/78; B33Y 40/20; B33Y 80/00

(57) ABSTRACT

A 3D printed prosthetic liner adapted to provide an interface between a residual limb and a prosthetic socket, specifically a prosthetic liner manufactured using a 3D printing device, the surface of which being treated with one or more applications of a casting resin, and preferably a urethane-based casting resin forming a treated prosthetic liner having enhanced strength and pliability.

### CROSS-REFERENCE TO RELATED APPLICATIONS

This International PCT application claims the benefit of and priority to U.S. Provisional Application No. 63/338,483 filed May 5, 2022, the specification, claims and drawings of which are incorporated herein by reference in their entirety.

### FIELD OF INVENTION

Generally, the inventive technology disclosed herein relates to the field of prosthetic devices, and more particularly improved 3D printed prosthetic liners adapted to provide an interface between a residual limb and a prosthetic socket.

### BACKGROUND

Traditional prosthetic devices typically operate by securing the residual limb into a rigid or semi-rigid socket. A socket may commonly refer to the portion of a prosthesis that fits around and secures a residual limb, and to which prosthetic components, such as a foot, are attached. Traditional prosthetic devices, such as sockets, are generally designed to stabilize the skeletal components of the residual limb and allow minimal relative movement between the socket and the residual limb. To effectively support the residual limb and allow for the efficient transfer loads from the residual limb to the ground, the prosthetic sockets are designed to provide a sufficient support to secure the residual limb within the socket, while at the same time allowing sufficient flexibility to allow for circulation and account for other physiological, temporal or environmental changes that may affect the shape and/or volume of the residual.

To increase comfort, a prosthetic liner may be positioned between the prosthetic socket and the residual limb. In general, the prosthetic liner comprises a closed base body with a proximal opening and is positioned over the residual limb. Such prosthetic liners provide a protective cover adapted to reduce movement and chafing between the skin of a residual limb and a hard prosthetic socket. Prosthetic liners are typically made from a flexible cushioning material and may include an inner portion or layer formed from a polymeric material. The polymeric material may serve as a cushioning material to offer stability and adhesion to soft-tissue of a limb. An outer cover may be provided in the form of a textile layer upon and/or to which the polymeric material or layer secures.

Traditional prosthetic liners are constructed from polymeric materials, preferably including elastomeric materials such as silicone, in part due to such materials being soft to the touch but resistant to pressure. Such traditional silicone prosthetic liners are ordinarily formed by providing an uncured silicone material in a liquid or semi-liquid form and then curing the silicone material within a set of molds, such that the cured silicone assumes the desired liner shape. Curing processes may involve the use of different catalysts, crosslinking agents, cure inhibitors, and accelerators. Other additives may be added to influence the durometer of the silicone liner and/or to provide other desired properties, such as skin-beneficial properties. However, due to the complexities of manufacturing and controlling the elastomer-curing processes, use of silicone prosthetic liners is expensive and can be difficult to customize to specific dimensions of an individual user's residual limb.

To overcome these limitations, the present inventors employ modern 3D printing techniques and material to generate custom prosthetic liners that are less expensive to create and can be more fully customized to conform to a user's residual limb. However, one drawback to the use of 3D printed prosthetic liners involves the semi-rigid materials used to generated the liners. There is thus a need for a prosthetic liner that incorporates the cost saving and customization of 3D printing systems, while also being sufficiently pliable to provide a more reliable and comfortable connection between a residual limb and a prosthetic socket.

### SUMMARY OF THE INVENTION

One aspect of the present invention includes novel methods for manufacturing and treating a prosthesis device, and in particular a 3D printed prosthetic liner. In one preferred aspect, the invention includes the manufacture of a prosthetic liner using a 3D printing device. The surface of the liner may be treated with one or more applications of a casting resin, and preferably a urethane-based casting resin forming a treated prosthetic liner.

### BRIEF DESCRIPTION OF THE DRAWINGS

FIG. 1: shows a 3D model of an exemplary liner (1) for a transfemoral prosthetic device in one embodiment thereof;

FIG. 2: shows an exemplary untreated 3D liner (2) for a transtibial prosthetic device in one embodiment thereof;

FIG. 3: shows an exemplary untreated 3D liner (2) for a transfemoral prosthetic device in one embodiment thereof; and

FIG. 4: shows an exemplary treated 3D) liner (3) for a transfemoral prosthetic device in one embodiment thereof.

### DETAILED DESCRIPTION OF THE INVENTION

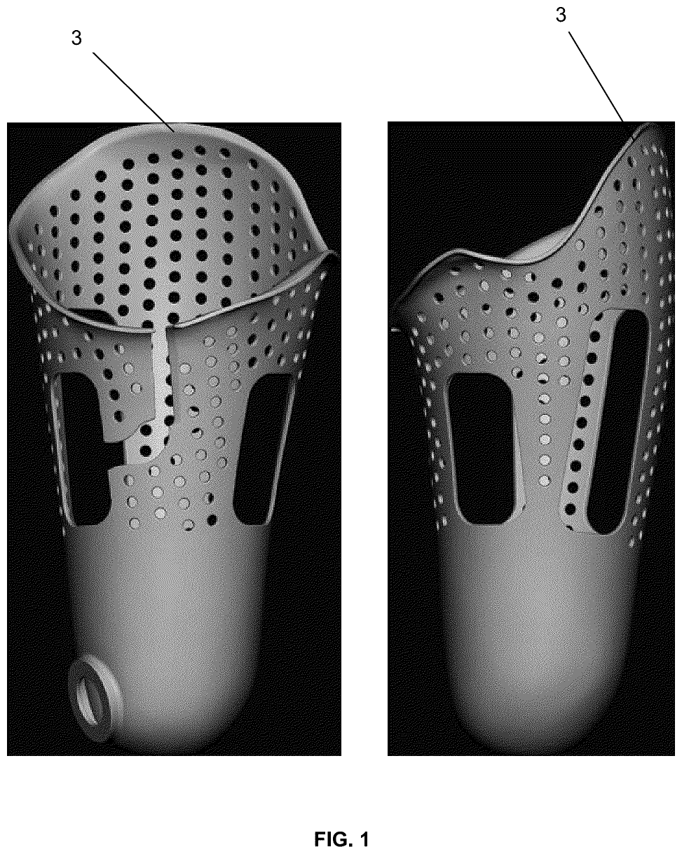

In one embodiment the invention may include a prosthetic socket configured to be capable of securing a prosthetic liner (1) through a socket interface. In a preferred embodiment a prosthetic liner of the invention may be configured to be secured over a residual limb. In a preferred embodiment this prosthetic liner of the invention may be configured to provide a buffer from a rigid prosthetic socket frame (not shown). In certain embodiments this prosthetic liner may be manufactured by a 3D printing device. As shown in FIGS. 1, the prosthetic liner includes a residual limb interface configured to confirm to the specific anatomical shape of a user's residual limb, and may further have an outer surface configured to be secured within the interior of the prosthetic socket frame or socket interface. In one preferred embodiment the residual limb interface may be configured to be closely mated with the user's residual limb providing support and shock adsorption, while the outer surface is configured to be mated with the socket interface on the user's prosthetic socket frame forming a fitted coupling. While initially described as disparate components, in certain embodiment both the socket frame and prosthetic liner may form an integral component. Additional embodiments not specifically shown may also include coupling devices on the prosthetic socket and/or prosthetic liner configured to secure the residual limb within the socket frame (2) during use.

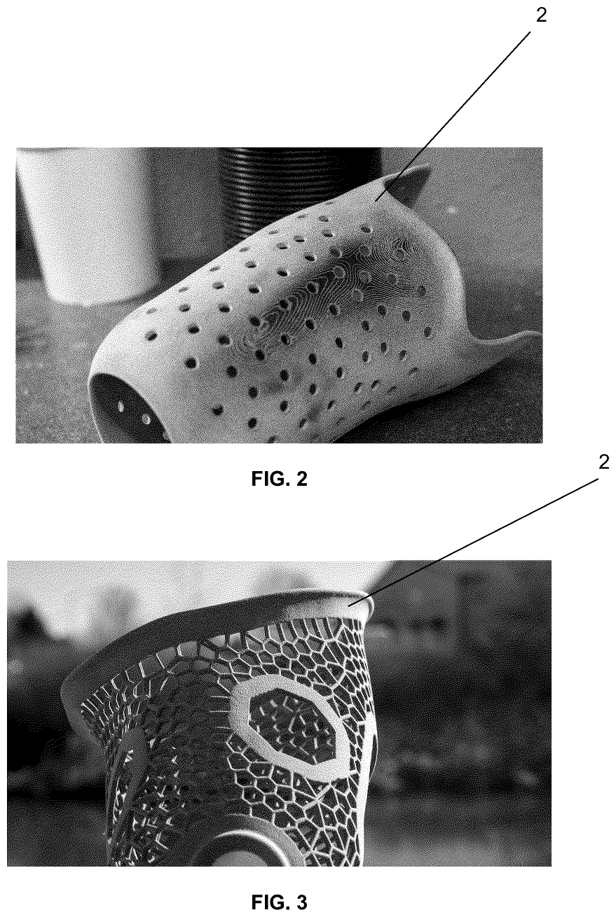

As shown in FIGS. 2-3, thermoplastic polyurethane (TPU) may be used as a base manufacturing material for the construction of a prosthetic liner using 3D production systems. In one specific embodiment, TPU, such as Lubrizol's Estane 3D M95A, may be used to manufacturer a 3D printed prosthetic liner. As noted above, traditionally fabricated flexible inner liners for prosthetic sockets are typically smooth and decrease the friction between the residual limb and the prosthetic socket. However, as shown in FIGS. 1-2, flexible liners generated by 3D printing processes using the TPU have a rough surface finish that actually leads to increased friction between the residual limb and the prosthetic socket. To eliminate this high-friction surface, the present inventors generate novel post-processing techniques to create a smooth, more uniform surface of 3D printed prosthetic liners.

In a preferred embodiment, a diagnostic evaluation of a patient in need of a prosthetic device may be performed and the initial three-dimensional shape of the residual limb may be digitally generated. The digital generation of this 3D model may be captured by invasive, or non-invasive diagnostic techniques known in the art. In a preferred embodiment, this 3D model of the residual limb may be digitally captured and communicated to a computer system that may further process the 3D model and generate a digital output for a customized 3D printed prosthetic liner configured to conform to the shape of the 3D model of the residual limb. In this embodiment, the digital output for a customized 3D printed prosthetic liner may be transmitted to a fabrication device or may be outputted into a CAD or other file format for automated mechanical or manual production. In another preferred embodiment, the computer system may upload the 3D model and generate a digital output for a customized 3D printed prosthetic liner that may further be input into a 3D fabrication device, such as a 3D printer. The 3D printing device may execute the 3D model file and fabricate a prosthetic liner configured to conform to the shape of the 3D model of the residual limb. Generally, a prosthetic liner generated by a rapid 3D printing/prototyping process may be made from a variety of materials, and preferably TPU as generally described above.

Residual TPU powder from the prosthetic liner or other 3D printed components of the invention may be removed, for example by a manual brush or the use of directed compressed air. Once all loose powder has been freed from the surface, the prosthetic liner is subjected to an abrasive surface particulate cleaning application. In a preferred embodiment, a pressurized glass bead media of medium grit may be used to abrade the surface of the liner of the invention. After this surface abrading treatment, the liner of the invention may again be cleaned, for example with compressed air to remove any remaining powders or particles.

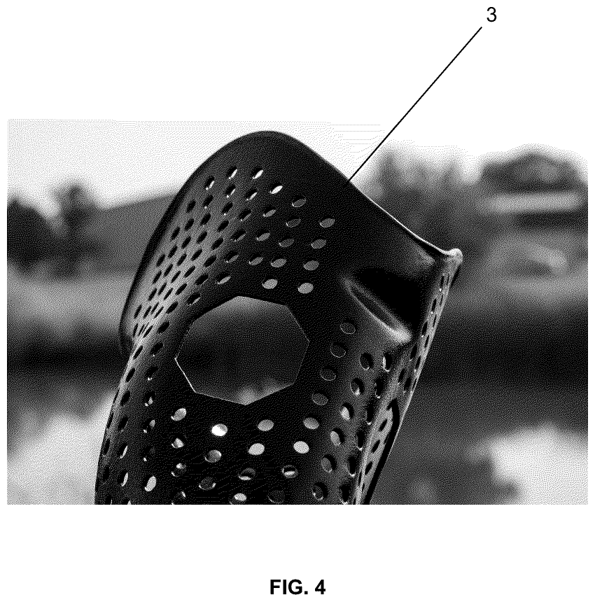

Next, a casting resin may be applied to the surface of the blasted and cleaned liner of the invention forming a treated prosthetic liner. Application of the casting resin may include manual application, for example by a brush, or through alternative means such as dipping or a pressurized casting resin spray. In a preferred embodiment, the casting resin of the invention may include a urethane casting resin. As described above, and generally showing in FIG. 2, the abrasive bead blasting step in post-processing creates a rough, inconsistent surface on the liner. The urethane casting resin counteracts these surface effects and as shown in FIG. 4, and creates a more consistent surface finish, which is difficult to achieve with the standard post-processing steps. Moreover, the urethane casting resin chemically interacts with the 3D printed TPU such that the treated prosthetic liner (3) becomes more pliant and flexible than an untreated 3D printed prosthetic liner (2). This enhanced characteristic has the benefit of reducing friction between the residual limb and hard-bodied prosthetic socket.

In still further embodiment, once the urethane resin coating has been set, the treated prosthetic liner maybe is dyed, for example using a black rit dye solution. After being removed from the dye, the treated prosthetic may further be sprayed with a silicone spray to ensure maximum frictional reduction between the patient and their liner.

As described above, the treated prosthetic liner of the invention may include one or more enhanced characteristics compared to untreated 3D printed liners. For example, the prosthetic liner and its various secondary components may exhibit a smoother and more uniform surface finish than before they were treated with a urethane-based casting resin. This smooth surface finish will reduce friction between the treated liner of the invention and a prosthetic sockets during use, and especially when a patient removes or attaches the socket to their residual limb. The treated liner of the invention further includes an enhanced feature of having additional structural strength. Specifically, as shown in FIG. 3, thinner, more delicate, and latticed parts of a treated prosthetic liner, become stronger and are less prone to breakages when abnormal forces are applied. This improved feature allows for the manufacturer prosthetic liners using 3D fabrication techniques using less material resulting in not only significant cost-savings, but allowing for prosthetic liners having thinner and less dense structures. Additionally, as noted above, treated prosthetic liners manufacturer of TPU are more pliant and flexible in three dimensions while retain their improved strength profile. Finally, the urethane resin coating of the invention allows for enhanced ability of the prosthetic liner to accept, retain and brightly display various dyes.

Naturally, all embodiments discussed herein are merely illustrative and should not be construed to limit the scope of the inventive technology consistent with the broader inventive principles disclosed. As may be easily understood from the foregoing, the basic concepts of the present inventive technology may be embodied in a variety of ways. It generally involves systems, methods, techniques as well as devices to accomplish a treated 3D printed prosthetic liner and the like. In this application, the methods and apparatus for the aforementioned systems are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention. As can be easily understood from the foregoing, the basic concepts of the present invention may be embodied in a variety of ways. It involves both techniques as well as devices to accomplish the appropriate system. In this application, the techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention may be described in some instances in method-oriented terminology, each element of the claims corresponds to a device and vice versa. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing the explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “support” should be understood to encompass disclosure of the act of “supporting”—whether explicitly discussed or not—and, conversely, were there effectively disclosure of the act of “supporting”, such a disclosure should be understood to encompass disclosure of a “supporting method and/or technique, and/or device” and even a “means for supporting.” Such changes and alternative terms are to be understood to be explicitly included in the description.

Any patents, publications, or other references mentioned in this application for patent, such as in the specification or an IDS are hereby incorporated herein by reference in their entirety. Any priority case(s) claimed by this application is hereby appended and hereby incorporated herein by reference in their entirety. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated herein by reference in their entirety. Finally, all references listed in the list of References To Be Incorporated By Reference In Accordance With The Patent Application or other information disclosure statement and the like filed with the application are hereby appended and hereby incorporated herein by reference in their entirety, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the methods and/or apparatus for providing a 3D printed prosthetic liner as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. It should be understood that this application also provides support for any combination of elements in the claims and even incorporates any desired proper antecedent basis for certain claim combinations such as with combinations of method, apparatus, process, and the like claims.

Any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The inventive subject matter is to include, but certainly not be limited as, a system substantially as herein described with reference to any one or more of the Figures and Description (including the following: for example, the process according to any claims and further comprising any of the steps as shown in any Figures, separately, in any combination or permutation).

Finally, Applicant reserves the right to seek additional design patent protection over the claimed invention; such that the drawings are fully enabled so as to allow one of ordinary skill in the art to know that the claimed design was in Applicant's possession at the time of filing. As such, it should be noted that any broken lines are to be included for the purpose of illustrating environmental matter and form no part of the claimed design should such become necessary.

1. A prosthetic system comprising:
a thermoplastic polyurethane (TPU) prosthetic liner; and
a coating resin applied to the surface of the liner forming a treated prosthetic liner.

2. The system of claim 1, wherein said prosthetic liner comprises a 3D printed prosthetic liner.

3. The system of claim 1, wherein said coating resin comprises a urethane-based coating resin.

4. The system of claim 1, wherein said prosthetic liner comprises a treated prosthetic liner selected from the group consisting of: a treated prosthetic liner for a transhumeral socket frame; a treated prosthetic liner for a transradial socket frame; a treated prosthetic liner for a transtibial socket frame; a treated prosthetic liner for a symes socket frame; a treated prosthetic liner for a hip disarticulation socket frame; a treated prosthetic liner for a knee disarticulation socket frame; and a treated prosthetic liner for a wrist disarticulation socket frame.

5. The system of claim 1, wherein said treated prosthetic liner comprises a treated prosthetic liner having a plurality of lattice structures.

6. The system of claim 1, and further comprising a prosthetic socket.

7. The system of claim 6, wherein said prosthetic socket is selected from the group consisting of: a transfemoral prosthetic socket; a transhumeral prosthetic socket; a transradial prosthetic socket; a transtibial prosthetic socket; a symes prosthetic socket; a hip disarticulation prosthetic socket; a knee disarticulation prosthetic socket; and a wrist disarticulation prosthetic socket.

8. The system of claim 1, wherein said treated prosthetic liner exhibits one or more of the following enhanced features compared to an untreated prosthetic liner:
increased strength;
increased pliability;
increase dye absorption; and
decreased friction with the residual limb.

9. The system of claim 1, wherein said coating resin is applied manually to said prosthetic liner.

10-22. (canceled)

23. A prosthetic liner device comprising a thermoplastic polyurethane (TPU) prosthetic liner, wherein the surface of the liner has been contacted with a coating resin forming a treated prosthetic liner.

24. The device of claim 23, wherein said prosthetic liner comprises a 3D printed prosthetic liner.

25. The device of claim 23, wherein said coating resin comprises a urethane-based coating resin.

26. The device of claim 23, wherein said prosthetic liner comprises a treated prosthetic liner selected from the group consisting of: a treated prosthetic liner for a transhumeral socket frame; a treated prosthetic liner for a transradial socket frame; a treated prosthetic liner for a transtibial socket frame; a treated prosthetic liner for a symes socket frame; a treated prosthetic liner for a hip disarticulation socket frame; a treated prosthetic liner for a knee disarticulation socket frame; and a treated prosthetic liner for a wrist disarticulation socket frame.

27. The device of claim 23, wherein said treated prosthetic liner comprises a treated prosthetic liner having a plurality of lattice structures.

28. The device of claim 23, and further comprising a prosthetic socket.

29. The device of claim 28, wherein said prosthetic socket is selected from the group consisting of: a transfemoral prosthetic socket; a transhumeral prosthetic socket; a transradial prosthetic socket; a transtibial prosthetic socket; a symes prosthetic socket; a hip disarticulation prosthetic socket; a knee disarticulation prosthetic socket; and a wrist disarticulation prosthetic socket.

30. The device of claim 23, wherein said treated prosthetic liner exhibits one or more of the following enhanced features compared to an untreated prosthetic liner:
increased strength;
increased pliability;
increase dye absorption; and
decreased friction with the residual limb.

31. A 3D printed prosthetic liner comprising:
a thermoplastic polyurethane (TPU) prosthetic liner, wherein the surface of the liner has been contacted with a coating resin forming a treated prosthetic liner; and
wherein said treated prosthetic liner comprises a treated prosthetic liner having a plurality of lattice structures.

32. The prosthetic liner of claim 31, wherein said coating resin comprises a urethane-based coating resin.

33. The prosthetic liner of claim 31, wherein said prosthetic liner comprises a treated prosthetic liner selected from the group consisting of: a treated prosthetic liner for a transhumeral socket frame; a treated prosthetic liner for a transradial socket frame; a treated prosthetic liner for a transtibial socket frame; a treated prosthetic liner for a symes socket frame; a treated prosthetic liner for a hip disarticulation socket frame; a treated prosthetic liner for a knee disarticulation socket frame; and a treated prosthetic liner for a wrist disarticulation socket frame.
